(19) United States
(12) Patent Application Publication
(10) Pub. No.: US 2009/0149721 A1
(43) Pub. Date: Jun. 11, 2009

(54) System for Monitoring, Analyzing and Auto-feedback of Health Conditions

(76) Inventor: Chang-Ming Yang, Miaoli (TW)

Correspondence Address: Dr. BANGER SHIA;Int''l Patent Office of Bang Shia, 102 Lindencrest Ct, Sugar Land, TX, 77479-5201, US

(21) Appl. No.: 11/718,096

(22) PCT Filed: Oct. 29, 2004
(86) PCT No.: PCT/CN04/01237

§ 371 Date: Apr. 27, 2007

(51) Int. Cl. A61B 5/00; A61N 1/00; A61F 7/00; A61N 7/00

(52) U.S. Cl. 600/301; 607/2; 607/96; 601/2

(57) ABSTRACT

The invention provides a system for monitoring, analyzing and auto-feedback of health conditions. The system can analyze the conditions of a user by monitoring the distribution of fat, muscle activities, and changes of poses. The system can further suggest or instructs the user to do suitable exercise and adjust to correct poses or behaviors, to reduce the accumulation of fat and to avoid the withering of muscles. The system can also analyze the psychological conditions to provide better assistance of health. Furthermore, the system includes a sensor, a near-end monitoring center, and a distal-end monitoring center. The sensor can detect the physical and psychological conditions of the user, and transfer related information to the near-end monitoring center, which can monitor, analyze, and display the conditions and either send feedback or provide instructions to the user. Additionally, the user can interact with the distal-end monitoring center via the near-end monitoring center.

### BACKGROUND OF THE INVENTION

1. Field of the Invention

The present invention relates to a system for auto-monitoring and analyzing health conditions, and more particularly, to a system, which includes a near-end monitoring center and a distal-end monitoring center, for monitoring, analyzing, and automatically feedback of health conditions. The system of the invention is capable of auto-monitoring continuously and bi-directly the health conditions of human body, mind, and daily life, and providing guides or suggestions to improve the health of human beings.

2. Description of the Prior Art

As time goes by, the environment of human beings becomes more and more comfortable, and our demand for the quality of life gets higher and higher. Moreover, with the development of medication, people's life expectancy is much longer than before.

However, to pursue better living conditions, people become very busy in their everyday life. However, most people need a regular daily life, so a hustle-bustling lifestyle, such as abnormal eating, can easily cause excessive pressure in human beings, other problems include poor sleeping quality, inability to relax, binge eating and excessive drinking, overloading work, and insufficient exercise.

Therefore, how to strike a balance between good life quality and wealth has become a major issue for studying.

In a word, the daily behavior of human beings mainly includes the changes in morale and the movement of limbs. Accordingly, the invention records the changes of morale and the movement of limbs, collects and analyzes the physical and psychological conditions of the user. Moreover, the system of the invention can provide suggestions to the user and interact with distal-end monitoring center.

### SUMMARY OF THE INVENTION

Accordingly, an aspect of the invention is to provide a system for auto-monitoring, analyzing and giving feedback of user's health conditions via continuous monitoring of the health conditions of an user, and providing guides or suggestions to improve the health of the user.

The system of the invention includes at least a sensor, disposed in a garment, for obtaining health conditions of an user; a near-end monitoring center, for receiving, analyzing, storing, and transferring data from the sensor or a distal-end monitoring center, and processing the data through figures, voices, images, texts, tactile sense, vibration, or 3D animations, and forward them to related personnel for determination, and controlling the sensor to detect repeatedly according to the user's health conditions; a distal-end monitoring center, for receiving the information provided by the near-end monitoring center, or transferring other information to the near-end monitoring center. The system asks the user to perform different poses, so as to monitor the muscles of different body portions of the user and store the conditions as basic data of the user. The system can further monitor the user and compare the monitoring conditions with the basic data every time to determine the conditions of the user's muscles. When the system detects an abnormal condition, such as an insufficient EMG index, which means insufficient exercise, it will suggest the user to increase the strength or times of exercise. On the contrary, when the system detects another abnormal condition, such as an over index of EMG, which means wrong extension of the body, wrong sitting or sleeping position, or using muscles in a wrong way, it will suggest the user to relax or adjust his or her position to improve the situation.

The method of the invention includes the step of analyzing the user's behavior by disposing the sensors on the user's garment to monitor the user's behavior. Because the muscles of human body have established organization and active area, incorrect behavior may cause improper utilization of muscles and may eventually injure human body. The recent medication has thoroughly studied the relationship between correct body movement and health, however, it is still hard to provide helpful suggestion to different person over a long period of time. Through the monitoring method of the invention, a personal body movement database for each individual user can be established. Moreover, the method of the invention can provide suggestions to the user according to the database, to improve the user's health.

Additionally, besides monitoring the user's body movement, the method of the invention can also monitor the distribution of fat in the user's body. Because of the bountiful material life, people nowadays may absorb too much fat or sugar while doing insufficient or wrong exercise, thus causes an imbalanced distribution of fat, which further causes a decrease of immunity in human body, which may result in serious disease, such as diabetes, heartache, and high blood fat. Through application of the system of the invention, the user can realize the distribution of fat in his or her own body, and further controls weight or modifies his/her body shape by doing correct exercise, calculating calories burned, or eating right under instructions.

Moreover, the factors that affect people's health further include the change of morale. Because the life step is too hectic, people may not be able to handle the pressure in life, and may lose their control of morale. This situation may further cause abnormal reactions such as depression or insomnia, and further affects our health. Accordingly, the method of the invention can also monitor user's changes of morale by detecting the voice, attention, and heartbeat of the user, with the options of the blood pressure and respiration of the user as well, to analyze the user's morale. Consequently, the method may suggest the user to relax at a proper time, to reach physical and psychological health.

Furthermore, the method of the invention can be applied to monitor daily life and changes of morale of expecting mothers, elder people, and patients with diabetes, obesity, amnesia, melancholia, and apoplexy, and provide suitable assistance and suitable rescue under critical conditions.

The advantage and spirit of the invention may be understood by the following recitations together with the appended drawings.

### BRIEF DESCRIPTION OF THE APPENDED DRAWINGS

FIG. 1 shows the disposition of the system of the invention;

FIG. 2 shows the disposition of the system of the invention;

FIG. 3 is a flow chart of the method of the invention;

FIG. 4 is a flow chart of the emergency rescue method of the invention; and

FIG. 5 is a flow chart of the emotion monitoring method of the invention.

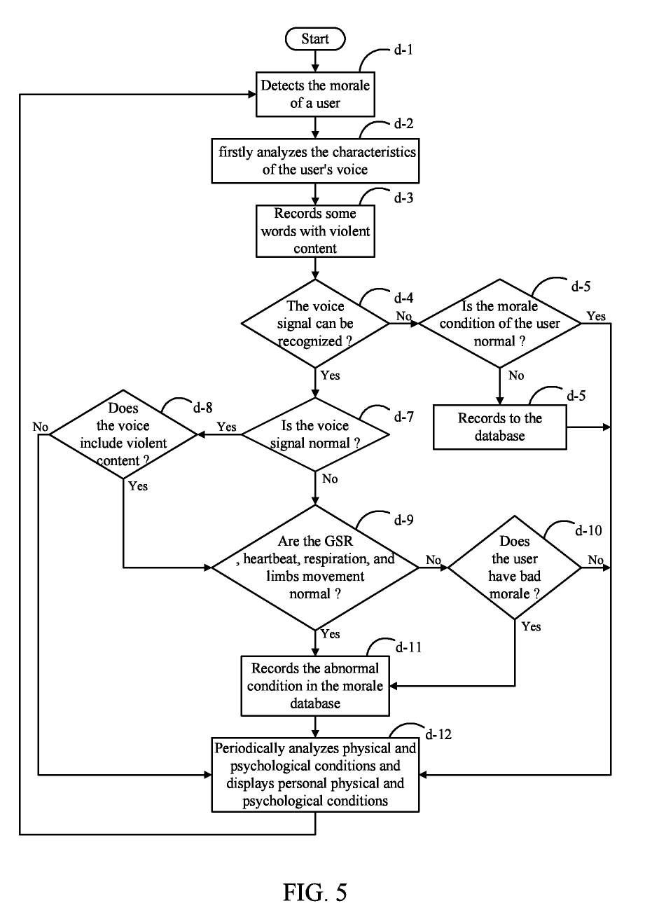

### DETAILED DESCRIPTION OF THE INVENTION

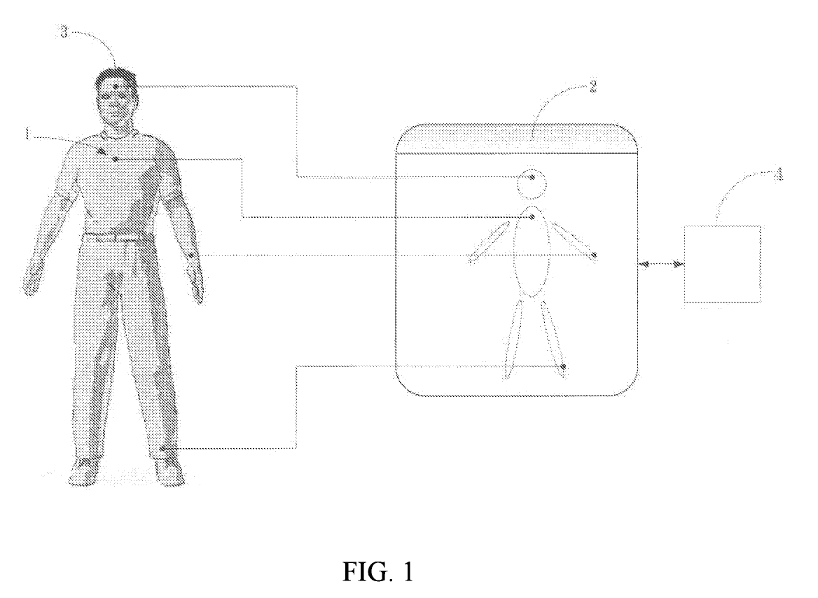

Please refer to FIG. 1, the system of the invention includes a plurality of sensors 1 disposed in the user's garment with different functions. Moreover, the sensor 1 of the invention can be fixed, detachably composed, or directly disposed on the user's garment, and can even be further embedded into human body. The sensor 1 can also automatically move its position to the near-end monitoring center 2, or can be moved by the user to a desired position for detecting the health condition of the desired position. After use, the sensor can be disassembled in case the garment needs to be washed. The system of the invention can be integrated with a garment for the user 3 to wear to perform monitoring and analysis (the garment includes, but not limited to, hats, masks, gloves, socks, and belts; and the desired monitoring positions include face, eyes, and other suitable positions). Furthermore, the sensor 1 of the present system includes, but is not limited to, a skin electric current reactor for detecting Galvanic Skin Reflex, an electromyogram sensor, an electrocardiogram sensor, a respiration sensor, a temperature sensor, a video camera, a body position sensor such as a mercury switch array, a pressure sensor for detecting the forced situation of the body, an accelerometer for detecting the activities of the body, and a heat flux sensor. Therefore, the system of the invention can detect the changes in the user's poses, the duration of each of the poses, distribution of fat, respiration rate, distribution or change of water in the user's body, and the heat flow. Because the above-mentioned sensors 1 have similar functions, we can easily detect several physical conditions withoutdispositing extra sensors. For example, we can obtain the distribution of fat and water through measuring the resistance between two points of skin surface; we can also obtain the reaction of muscles or sensitivity of nerves; and, on the other hand, we can awake the user through electronic stimulation or massage his muscles by TENS.

The monitoring data of the above-mentioned sensors 1 will be transferred to a near-end monitoring center 2, disposed in the garment or in a communication equipment (e.g., PDA, mobile phone, and computer, etc.), for receiving, analyzing, storing, and transferring data from the sensor 1 or a distal-end monitoring center. If the near-end monitoring center 2 is disposed in a communication equipment, users in the same environment, such as husband and wife, or colleagues, can share the same near-end monitoring center 2. Moreover, a user can change the near-end monitoring center 2 when he is in a different environment, for example, when the user is at home, he or she can use the computer as the near-end monitoring center, whereas when the user is outside, he or she can use the mobile phone as the near-end monitoring center 2. The near-end monitoring center 2 can receive, analyze, store, and transfer data from the sensor or a distal-end monitoring center, and perform the data through figures, voices, images, texts, tactile sense, vibration, or 3D animations, to the user 3. If needed, the near-end monitoring center 2 can communicate with a distal-end monitoring center 4. Moreover, medical personnel, recovery agents, psychologists, and dietitians at the distal-end can determine the results and communicate with each other, and instruct the user 3 to exercise or adjust his or her poses and life behavior via the system. The system can periodically display the distribution of fat in the user's body, and suggest the user ways to remove or reduce the fat via 3D animation, images, voices, texts, vibrations, and tactile sense, and can further detect if the user 3 exercises correctly, and periodically display the results of calories gained or burned after eating or exercising. Moreover, after daily work, the system can display the activities of muscles and the amount of calories burned, and if there is something abnormal, the system can warn the user 3, and send feedback of the changes for suitable adjustment. Furthermore, combined with the video camera as mentioned above, the system can monitor the content of foods, and further suggest the user 3 to choose the correct food to eat. The user can interact with the near-end monitoring center or the distal-end monitoring center by button, voice, image, and text.

Furthermore, the invention can also be applied during sleeping, for monitoring and analyzing time for bed, time for falling asleep, period of sleep, conditions of muscles, respiration, and heartbeat during sleep, to understand the user's sleeping quality and provide suitable suggestions, such as drug treatment or behavior therapy. Moreover, the behavior includes: 1. sleep hygiene—warning the user to go to bed on time, wake up on time, do some boring things if the user can not fall asleep 20 minutes after going to bed; 2. relaxation therapy—instructing the user to reach progressive muscle relaxation and feedback the effect of user's muscle, or instructing the user to meditate, imagine, and biofeedback; 3. stimulus control therapy—avoid waking up the user by suggesting changes of behavior; 4. chronotherapy—applying voice, text, image, vibration, and tactile sense to suggest the user to adjust the time for going to bed; and 5. light therapy—suggesting the user receiving light in the early morning or evening to change the speed of the biological clock of the user. The system can instruct the user via the above-mentioned image, voice, text, vibration, tactile sense, and 3D animation, and send back feedback for analysis. If the condition could not be improved, the system will suggest the user to contact medical personnel or transfer related data to the distal-end monitoring center 4.

In addition, the system of the invention can further include a video camera for providing real-time image to the near-end monitoring center or the distal-end monitoring center 4 to analyze the user's physical conditions.

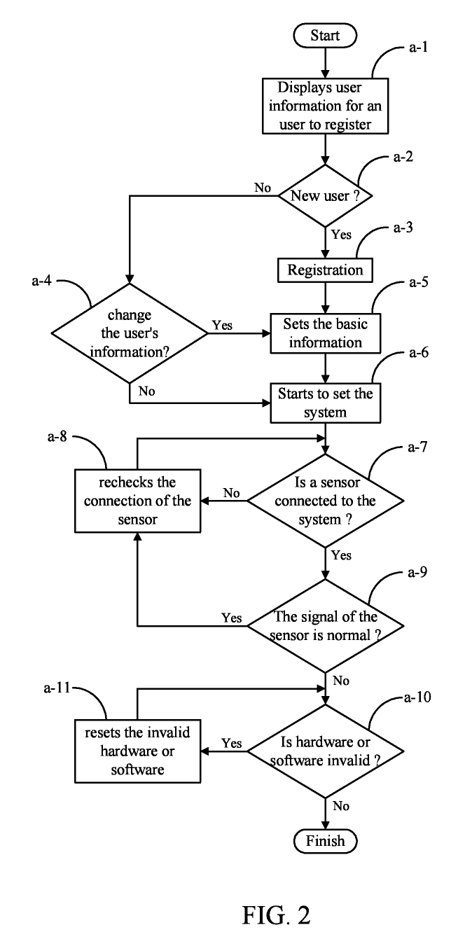

As shown in FIG. 2, the monitoring system of the invention needs some settings, and the setting steps are as follows:

a-1 the system displays user's information for an user to register;

a-2 the system determines if the user is a new user;

a-3 if the user is a new user, performs a registration process;

a-4 if the user is not a new user, asks the user if he or she wants to change the user's information;

a-5 sets the basic information, such as height, weight, age, medical history, and contact information in case of emergency, such as the phone number, mobile phone number, e-mail address, MSN, and ICQ identity;

a-6 starts to set the system;

a-7 checks if a sensor is connected to the system, if no, process step a-8, and if yes, process step a-9;

a-8 rechecks the connection of the sensor;

a-9 tests if the signal of the sensor is normal, if no, recheck the sensor, and if yes, process step a-10;

a-10 tests if any hardware or software is invalid, if yes, process step a-1;

a-11 resets the invalid hardware or software.

After the above-mentioned setting process, the system of the invention can normally perform monitoring.

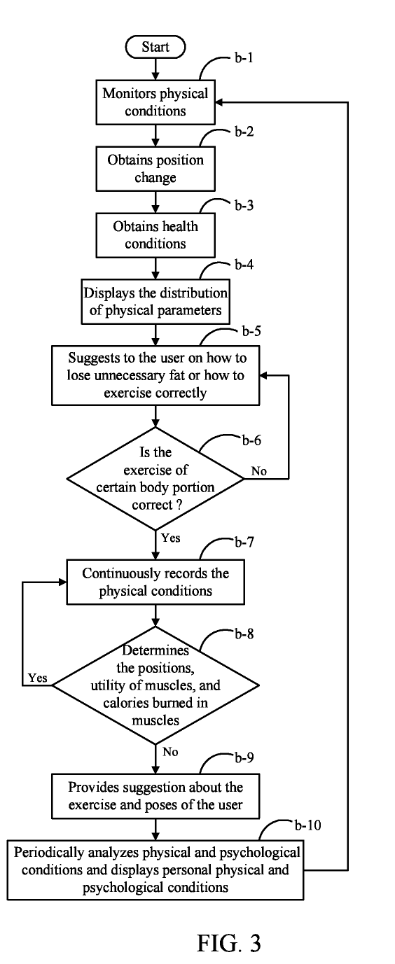

As shown in FIG. 3, a general application is described as below:

b-1 monitors physical conditions;

b-2 obtains position change of the user at pre-determined time or randomly determined time to be an accordance to instruct the user;

b-3 obtains health conditions such as the strength and utility rate, the distribution of calories burned, body fat, water, the electromyogram, and heat flow of muscles in different body portions, to be an accordance to instruct the user;

b-4 displays the distribution of physical parameters related to each of the poses through figures, voices, images, texts, tactile sense, vibration, or 3D animations, to the user and related medical personnel;

b-5 suggests to the user on how to lose unnecessary fat or how to exercise correctly through figures, voices, images, texts, tactile sense, vibration, 3D animations, or electronic stimulation;

b-6 monitors if the exercise of certain body portion is correct, if not, returns to step b-5, and if yes, process step b-7;

b-7 continuously records the physical conditions for accumulating and compiling statistics of the data;

b-8 determines the positions, utility of muscles, and calories burned in muscles of different body portions, and if the utility of muscles is correct, returns to step b-7 for continuous recording;

b-9 provides suggestion about the exercise and poses of the user through figures, voices, images, texts, tactile sense, vibration, 3D animations, or electronic stimulation, and traces the effects;

b-10 periodically analyzes physical and psychological conditions and displays personal physical and psychological conditions, for example, displays depression inventory to reflect the depression level of the user; display the results of Mini-Mental State Examination to reflect the intelligence condition of the user, and suggests a user with melancholia to listen to music, and traces the user's condition for a long period, and if needed, contacts with a distance psychologist or doctor.

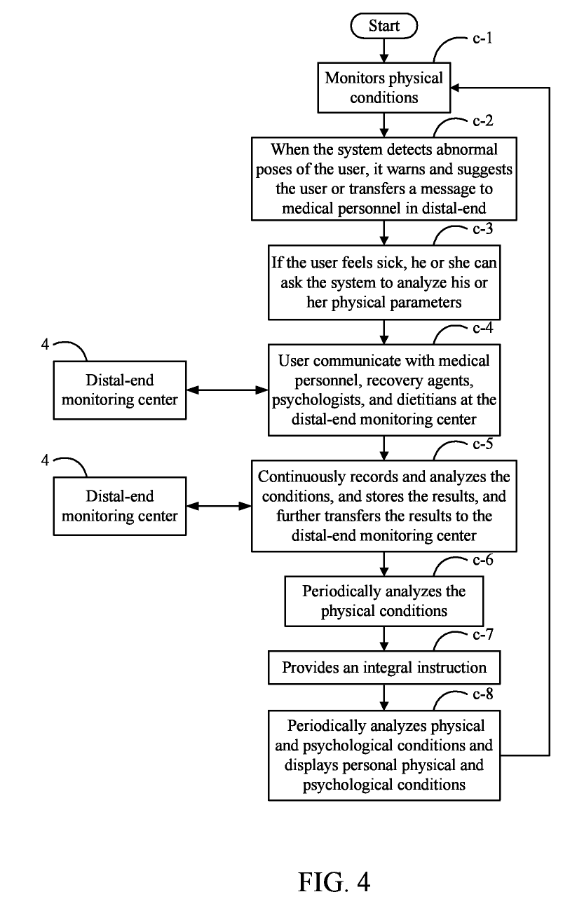

As shown in FIG. 4, the invention can further provide more active medical assistance, which is described as follows:

c-1 performs the monitoring of physical conditions;

c-2 when the system detects abnormal poses of the user, it warns and suggests the user or transfers a message to medical personnel in distal-end for help, for example, when the system detects the user has wrong poses or maintains the same pose for too long, it will warn the user to adjust his or her pose; furthermore, when the system detects increased blood pressure, respiration, heartbeat, and serious change of body temperature, it will warn the user and further send the message to the medical personnel at the distal-end monitoring center for help;

c-3 if the user feels sick, he or she can ask the system to analyze his or her physical parameters such as EMG by ordering through pressing buttons, texts, or speaking, so as to find the reason why he or she feels sick, and the system feedbacks the monitoring results to the user to improve the situation. The system can supply medical assistance as well, such as cold or hot compression, ultrasonic massage, and TENS;

c-4 the user can communicate with medical personnel, recovery agents, psychologists, and dietitians at the distal-end monitoring center 4 to obtain better assistance, similarly, the medical personnel can provide instructions to the user via the system when they observe abnormal signals;

c-5 continuously records and analyzes the conditions, and stores the results, and further transfers the results to the distal-end monitoring center 4 such as a hospital to establish personal physical reference data, which can be combined with the user's physical examination report, and then the medical personnel can give an instruction to the user via the near-end monitoring center 2;

c-6 periodically analyzes the physical conditions, such as distribution of fat, exercise conditions, changes of calories burned, daily life, behavior, sleeping quality, weight, blood pressure, and respiration;

c-7 provides an integral instruction, which focuses on, such as eating, sleeping, exercise, to the user, and further performs treatment such as cold/hot compression, or massage, and informs the user of the correct information through figures, voices, images, texts, tactile sense, vibration, or 3D animations, and if the user exercises in the wrong way or makes wrong movement, the system can warn the user to make adjustment.

In practice, the system of the invention can be applied to continuously monitoring patients of many diseases, such as asthma, pulmonary tuberculosis, or drug abuse, and provide good suggestions or instructions. For instance, when the system detects the user continuously coughing for two to three months, the near-end monitoring center 2 can suggest the user to perform an X-ray examination or to test his or her sputum for the diagnosis of pulmonary tuberculosis. The near-end monitoring center 2 can continuously transfer the monitoring results to the distal-end monitoring center 4 for close monitoring. For another example, the system can be applied to a segregated patient such as a SARS patient, the near-end monitoring center 2 can monitor the change of body temperature and daily life of the patient, if it detects any abnormal condition, it can transfer the results to the distal-end monitoring center 4. Similarly, the system can be applied to continuously monitor a drug taker and transfer the monitoring results to the distal-end monitoring center 4. In other words, the system of the invention can also be applied on a parolee or segregated patient to prevent crimes or accidents. The invention can further be applied on an animal to monitor the health conditions of the animal.

Additionally, the present invention can be applied to monitor changes of morale, and provide information to assist a psychologist or a doctor to treat the user. The steps for monitoring the morale of the user are described as follows:

d-1 detects the morale of a user;

d-2 if the user is a new user, the system firstly analyzes the characteristics of the user's voice, such as volume, tone, timbres, with the background sounds such as sounds from within houses, factories, and roads;

d-3 at the same time, the system records some words with violent content, such as “I am happy”, “sad”, and “don't want to live”, and other words related with physical conditions, such as “headache”, and “stomachache”, and combines with the user's voice to establish a personal morale database for the user;

In detail, the voice can be input by the user or the near-end monitoring center 2 or the distal-end monitoring center 4, to establish the morale database, and the depression score and result of MMSE can be added to the database to increase the accuracy of determination;

d-4 checks if the voice signal can be recognized;

d-5 if the voice signal can not be recognized, the near-end monitoring center 2 asks the morale condition of the user;

d-6 if the user's morale condition is abnormal, the near-end monitoring center 2 classifies the condition into “happy” or “unhappy”, and records to the database;

d-7 if the voice signal can be recognized, the near-end monitoring center 2 determines if the voice signal is normal;

d-8 if the voice signal is normal, the near-end monitoring center 2 further determines if the voice includes violent content, and if the voice does not include violent content, the near-end monitoring center 2 determines the condition of the user is normal;

d-9 if the voice signal is abnormal or the voice includes violent content, the near-end monitoring center 2 further determines if the GSR, heartbeat, respiration, or limbs movement are normal;

d-10 if the GSR, heartbeat, and respiration are normal, the system further asks the user if he or she has bad morale, and if the answer is yes, the system records the abnormal condition in a morale database;

d-11 if any of the GSR, heartbeat, or respiration is abnormal, the system directly records the abnormal condition in the morale database, moreover, the times and duration of bad morale are also be recorded;

d-12 after the above-mentioned steps, the system periodically analyzes physical and psychological conditions and displays personal physical and psychological conditions, for example, displays depression inventory to reflect the depression level of the user; display the results of Mini-Mental State Examination to reflect the intelligence condition of the user, suggests a user with melancholia to listen to music, traces the conditions for a long period of time, and if needed, contacts with a distance psychologist or doctor.

Furthermore, in regard to a noisy environment, poor sleeping quality, or other emergent conditions, the method of the invention includes the steps of:

a. records the duration in noisy environment for the user to improve;

b. records the times, volume, and timbres of daily speaking, and displays or suggests improvement through text, voice, image, 3D animation, and vibration, if the condition can not be improved, the system is capable of communicating with a distance medical personnel; furthermore, if the system detects that the user develops apnea, it can record the condition, and warns the user through sound, electronic stimulation, or vibration;

c. when the system detects pre-determined words, such as “suicide”, “don't want to live”, “want to die”, or abnormal behaviors, such as quietness for a long time, falling over, yelling or crying, it can inform the distal-end monitoring center 4 immediately;

d. determines the times and duration of eating from the EMG of throat portion or sounds, or by asking the user;

e. determines the duration of sleeping from the poses or sleeping reaction, and analyzes changes of respiration, heartbeat, muscle conditions, sleeping duration, and bedding time, so as to determine the sleeping quality and provide an instruction to the user;

f. if the system detects stridor, it can warn the user immediately and check if the user is under asthma condition, and if yes, the system suggests the user to take medicine, and if the condition is not improved, the system further communicates with the distal-end monitoring center 4.

Through the morale monitoring mechanism, the system can determine the psychological conditions of the user and display the conditions to the user, and then optionally suggests that the user listens to music, or provide a massage by TENS, ultrasonic waves, or provide cold or hot compression to the user, or assist medical personnel, family members, and psychologist at distal-end to take care of the user. Moreover, when the depression index is above a standard value, or when the user wants to talk to someone, the near-end monitoring center 2 can provide 3D images of real or virtual person, photos, music, texts, or voices to interact with the user. The medical personnel at the distal-end monitoring center 4 can talk to the user via video conference to reconcile depression of the user. Moreover, the system of the invention can include a plurality of distal-end monitoring centers, and the user can chose a suitable or nearest center that provides the service. The plurality of distal-end monitoring centers can communicate with each other to provide an optimal solution.

The invention can further help maintain good health of an expecting mother. In detail, the system can monitor, analyze, and suggest expecting mother via the steps as described above, so as to allow the expecting mother to utilize suitable strength and position of exercise. As mentioned above, the system of the invention can also monitor the movement and heartbeat of a fetus. Moreover, the system can also be applied to monitor the physical conditions of patients with diabetes, obesity, amnesia, melancholia, and apoplexy, and suggest the patients to contact the medical personnel in the distal-end when the system detects an emergency condition. Additionally, the near-end monitoring center 2 can also interact with other equipments in the same environment, for example, when the system determines the body temperature is excessive during sleeping, it can automatically control the temperature setting of an air conditioner or inform a person to adjust the setting manually. For another example, when the system detects a leak of fuel gas, the near-end monitoring center 2 can warn the user.

The system and method for monitoring and analyzing physical conditions of the invention can scientifically monitor the physical and psychological conditions of a user, and analyze these conditions. Furthermore, the system of the invention is capable of auto-monitoring continuously and bi-directly the health conditions of human body and mind as well as daily life, and providing guides or suggestions to improve the health of the human beings.

With the example and explanations above, the features and spirits of the invention are hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. A system for monitoring, analyzing and auto-feedback a health condition, comprising:
a sensor, fixed or detachably composed in a garment, for detecting health conditions of a user, and the sensor being capable of moving to a near-end monitoring center automatically, and the sensor being capable of being moved by the user to a desired position for detecting the health condition of the desired position;
the near-end monitoring center, disposed in the garment or in a communication equipment, for receiving, analyzing, storing, and transferring data from the sensor or a distal-end monitoring center, and performing the data through figures, voices, images, texts, tactile sense, vibration, or 3D animations, to related personnel for determination, who then controlling the sensor to detect repeatedly according to health conditions; and
the distal-end monitoring center, for receiving the information provided by the near-end monitoring center, or transferring other information to the near-end monitoring center.

2. The system of claim 1, wherein the garment is a hat, a glove, a sox, and a belt, worn by the user.

3. The system of claim 1, wherein the sensor is selected from the group consisting of: a sound sensor, a skin electric current reactor, an electromyogram sensor, an electrocardiogram sensor, a respiration sensor, a temperature sensor, a video camera, a body position sensor, a pressure sensor, an accelerometer, and a heat flux sensor; wherein the galvanic skin reflex (GSR) sensed by the skin electric current reactor is replaced by the observation of respiration and heart rate.

4. The system of claim 1, wherein the near-end monitoring center is directly disposed in the garment, or disposed in a PDA, a computer, or a mobile phone; wherein the near-end monitoring center is capable of being changed by the user, and performing GPS function.

5. The system of claim 1, wherein the sensor is directly embedded into human body.

6. The system of claim 1, further comprising a video camera for providing real-time image to the near-end monitoring center or to the distal-end monitoring center to analyze the user's daily life.

7. The system of claim 1, wherein the sleeping time and quality of the user is capable of being monitored and analyzed for a long period of time, if apnea happened, the near-end monitoring center being capable of recording the apnea and warning the user via voice, vibration, or electronic stimulation, and transferring a message regarding the apnea to the distal-end monitoring center.

8. The system of claim 1, further comprising a TENS device, an ultrasound device, and a cold/hot compression device are disposed on the garment.

9. The system of claim 1, wherein the garment system is capable of detecting external sound and sign language for the listening-impaired patients, and performing the sound and sign language through figures, voices, images, texts, tactile sense, vibration, or 3D animations, and self-pronouncing to communicate with the outside world.
